(19) United States
(12) Patent Application Publication
(10) Pub. No.: US 2019/0196668 A1
(43) Pub. Date: Jun. 27, 2019

(54) IMAGE DISPLAY SYSTEM AND IMAGE DISPLAY TERMINAL

(71) Applicant: KONICA MINOLTA, INC., Tokyo (JP)

(72) Inventor: Naotoshi SUGEGAYA, Tokyo (JP)

(21) Appl. No.: 16/227,670

(22) Filed: Dec. 20, 2018

(30) Foreign Application Priority Data
Dec. 25, 2017 (JP) 2017-247216

(51) Int. Cl. G06F 3/0482; G06F 3/0488; G16H 30/20

(52) U.S. Cl. CPC G06F 3/0482; G06F 2203/04803; G16H 30/20; G06F 3/04886

(57) ABSTRACT

An image display system including: an external device which stores a radiologic image; and a display terminal which displays the radiologic image on a display screen of a touch panel, wherein the display terminal has, on the display screen, an image display area for display of the radiologic image and a related examination list display area for display of a related examination list to select the radiologic image, and contents of a plurality of related examinations in the related examination list are chronologically displayed in order based on a screen touch operation on the display screen of the touch panel.

### CROSS-REFERENCE TO RELATED APPLICATION

The present application claims priority under 35 U.S.C. § 119 to Japanese Patent Application No. 2017-247216, filed on Dec. 25, 2017, the entire contents of which are incorporated herein by reference.

### BACKGROUND

### Technological Field

The present invention relates to an image display system and an image display terminal.

### Description of the Related Art

Patent document 1 (Japanese Patent Application Laid Open Publication No. 2004-305272), for example, discloses a conventional radiologic interpretation system that displays radiologic images to be interpreted and other images taken in the past as references on one display screen. The radiologic system in Patent document 1 displays thumbnails of the images taken in the past on the display screen to facilitate the selection of an image taken in the past, and the image can be selected as a reference by a so-called drag and drop operation.

Due to the demand for urgent radiologic interpretation, needs for radiologic interpretation by radiologists outside the hospital are increasing. In that case, radiologic images should be interpreted on a compact mobile terminal instead of a large monitor of a display screen in an in-hospital system. If the layout displayed on an in-hospital monitor is displayed on such a compact terminal, the size of the display screen is limited, thus causing inconvenience in use, which is a problem in urgent radiologic interpretation.

### SUMMARY

An object of the present invention is to facilitate radiologic interpretation which is convenient even on a compact terminal.

To achieve at least one of the abovementioned objects, according to an aspect of the present invention, an image display system reflecting a first aspect of the present invention is characterized in including an external device which stores a radiologic image; and a display terminal which displays the radiologic image on a display screen of a touch panel, and that the display terminal has, on the display screen, an image display area for display of the radiologic image and a related examination list display area for display of a related examination list to select the radiologic image, and contents of a plurality of related examinations in the related examination list are chronologically displayed in order based on a screen touch operation on the display screen of the touch panel.

An image display terminal reflecting a second aspect of the present invention is characterized in including a display that displays a radiologic image, which is received from an external device, on a display screen of a touch panel; and an image display area for display of the radiologic image and a related examination list display area for display of a related examination list to select the radiologic image, on the display screen, and that contents of a plurality of related examinations in the related examination list are chronologically displayed in order based on a screen touch operation to the related examination list display area.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a block diagram illustrating a configuration of a display terminal;

FIG. 2 illustrates an exemplary view on the display screen of the display terminal;

FIG. 3 illustrates an exemplary view on the display screen of the display terminal;

FIG. 4 illustrates an exemplary view on the display screen of the display terminal;

FIG. 5 illustrates an exemplary view on the display screen of the display terminal;

FIG. 6 illustrates an exemplary view on the display screen of the display terminal;

FIG. 7 illustrates an exemplary view on the display screen of the display terminal;

FIG. 8 illustrates an exemplary view on the display screen of the display terminal;

FIG. 9 illustrates an exemplary view on the display screen of the display terminal; and

FIG. 10 illustrates an exemplary view on the display screen of the display terminal.

### DETAILED DESCRIPTION OF THE EMBODIMENTS

Hereinafter, one or more embodiments of the present invention will be described in detail with reference to the drawings. In the following embodiments, technically preferred features for the present invention are described. However, the scope of the invention is not limited to the disclosed embodiments or illustrated examples.

The configuration will now be described.

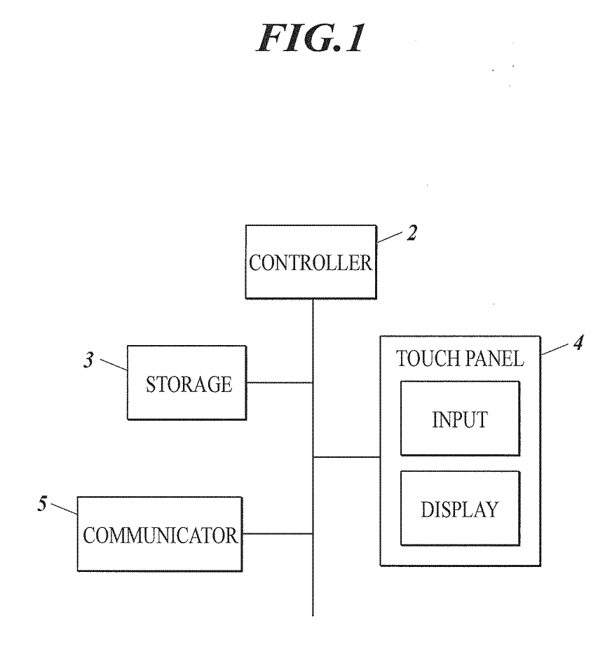

FIG. 1 illustrates the overall configuration of a display terminal 1 (image display terminal 1) according to the embodiment.

The display terminal 1 is portable, includes a touch panel, such as smartphone or tablet computer, and is communicatively connected to an external device via a network. The portable display terminal 1 is compact and is thus suitable for carrying by a person. The display terminal 1 according to the present embodiment is a tablet computer.

It should be noted that the network refers to an information communication network including, for example, the Internet, a wide area network (WAN), a local area network (LAN), a dedicated line, or a combination thereof.

As illustrated in FIG. 1, the display terminal 1 includes a controller 2, a storage 3, a touch panel 4, and a communicator 5.

The controller 2 includes, for example, a CPU and a RAM. The CPU of the controller 2 reads various programs stored in the storage 3, such as system programs and processing programs, develops the read programs in the RAM and executes various processes under the developed programs.

The storage 3 includes, for example, a non-volatile memory of a semiconductor. The storage 3 stores system programs and various programs necessary for the operation of the display terminal 1 in the present embodiment.

The touch panel 4 serves as both an input component and a display, detects the position of a touch operation by a user, such as a medical doctor or radiographer, with an operator, for example, user's finger or a stylus and receives the coordinate data. In the present embodiment, the touch panel 4 is of, for example, a capacitive sensing type or resistive type. Alternatively, any other type of touch panel may be used.

Upon detecting a touch operation on the touch panel 4, the controller 2 determines details (types) of a touch operation, i.e., whether a touch operation indicating a position on the display screen of the touch panel 4 (terminal screen) was performed, whether a touch operation indicating change of display contents displayed on the display screen (terminal screen) was performed, and whether a touch operation indicating entry of a command or data was performed.

The communicator 5 includes an interface for transmission and reception of various types of data, such as medical image data (radiologic images for interpretation), and various commands for related examinations, which will be described below, to/from the external device via wireless communication, for example, Bluetooth (registered trademark) and Wi-Fi. The display terminal 1 can access not only the specified external device but also any other external server and site via the communicator 5.

Although not illustrated, the display terminal 1 may include an appropriate power source, an external connection component (wired interface), a voice inputting component, and a voice outputting component besides the controller 2 as required.

Furthermore, internal devices, such as the controller 2 and the storage 3, are accommodated between the touch panel 4 and an exterior housing 6 holding the touch panel 4. The exterior housing 6 is provided with operational components for operating the internal devices, such as buttons (not shown), as required.

The display terminal 1 having such a configuration can display a radiologic image to be interpreted (hereinafter referred to as merely radiologic image) received from the external device on the display screen of the touch panel 4 in response to the controller 2 and a program stored in the storage 3. The display terminal 1 transmits and receives data, such as radiologic images, for related examinations to/from the external device in this manner and serves as an image display system.

Although not illustrated, the external device is an in-hospital server for management of patient data in, for example, a hospital or a clinic. A user usually utilizes an in-hospital client terminal to manage and use the patient data on a large in-hospital monitor. The display terminal 1 can access the external device which is an in-hospital server from outside the hospital to manage and use necessary information.

One example of the information that can be managed and used by the display terminal 1 is a medical image (radiologic image) for a related examination. Related examinations include a specific examination at a certain date and examinations linked to the specific examination, for example, an examination performed for a patient at a certain date and examinations performed for the same patient before and after the date.

The medical images for related examinations (radiologic images) stored in the storage of the in-hospital server are searched by accompanying information. If a medical image includes accompanying information, for example, at least one of the patient name, patient ID, and examination site is identical and the date of an examination is earlier or later than the certain date, the medical image can be determined to be a medical image of a related examination (radiologic image).

The certain date generally refers to a date that is not specifically identified and may be today or any day in the past. In the present embodiment, the dates of the past examinations are known; hence, the certain date refers to any one of examination dates of the patient.

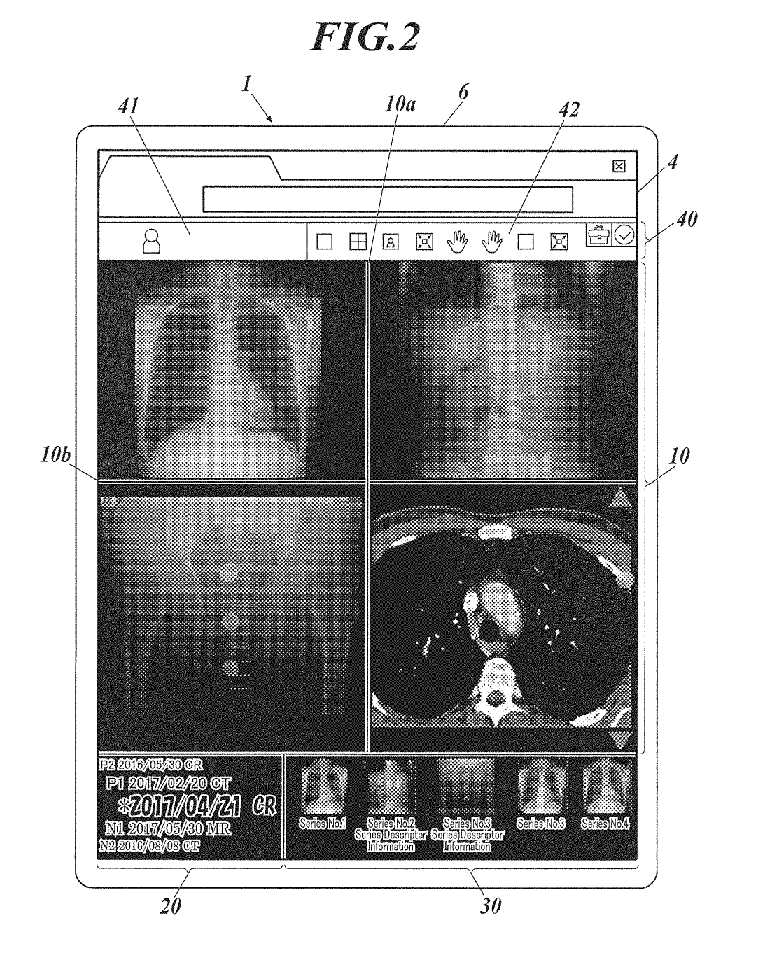

As illustrated in FIG. 2 to FIG. 7, the display terminal 1 presents an image display area 10 for display of radiologic images, a related examination list display area 20 for display of a related examination list and for selection of a radiologic image, a thumbnail display area 30 for display of thumbnails corresponding to the related examination list, and a function area 40 on the display screen of the touch panel 4.

The image display area 10 occupies the largest area on the display screen and exhibits radiologic images received from the in-hospital server which is an external device.

The image display area 10 according to the present embodiment is partitioned into four portions by boundaries 10a and 10b, and these portions exhibit different radiologic images.

Within the image display area 10, the boundaries 10a and 10b may be shifted and images may be displayed or hidden. The number of boundaries 10a and 10b may be increased or decreased to vary the partitioning of the image display area 10. This enables a user to arbitrarily vary the layout of the image display area 10.

When the image display area 10 exhibits radiologic images, the user needs to designate the examination performed at the certain date as a reference. In the present embodiment, Apr. 21, 2017 is the date of the examination designated by the user (hereinafter referred to as “the present examination”) as illustrated in FIG. 2.

In other words, in order to exhibit the radiologic images in the image display area 10, the user needs to select the present examination as a reference. The display terminal 1 can simultaneously receive the radiologic images of the present examination selected by the user and the radiologic images of other examinations associated with the present examination from the in-hospital server which is the external device.

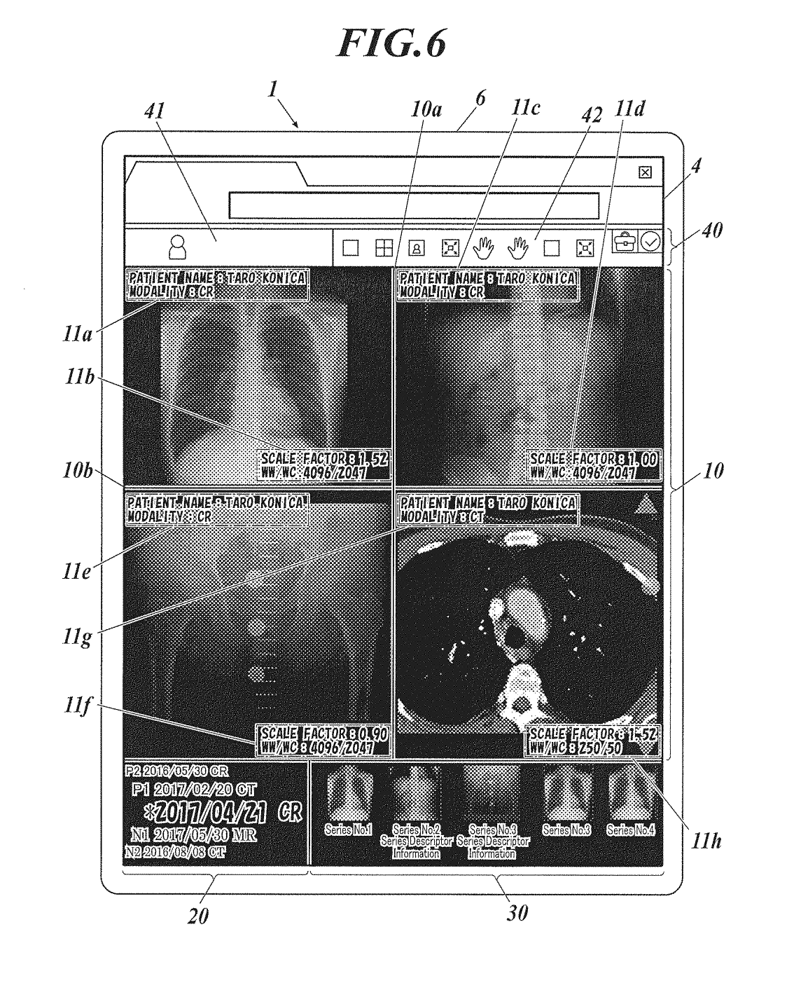

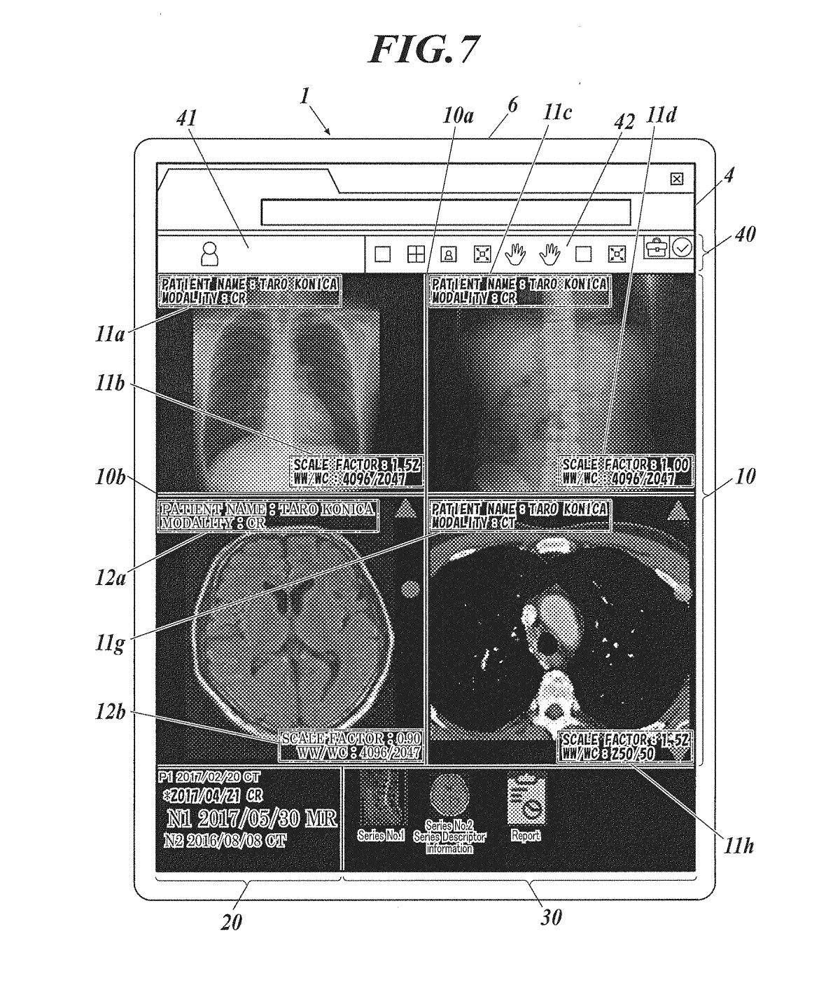

As illustrated in FIG. 6 and FIG. 7, the image display area 10 can exhibit not only radiologic images but also pieces 11a to 11h, 12a, and 12b of accompanying information on the radiologic images.

Examples of the pieces 11a to 11h, 12a, and 12b of accompanying information include, but is not limited to, patient name, patient ID, examined site of patient, date and time of examination, type of modality, and enlargement factor of radiologic image.

The modality refers to the type of imaging device that captures an image appearing on the image display area 10. Examples of imaging devices include a radiographic imaging device (a flat panel detector), a mammography device, a computed tomography (CT) device, a magnetic resonance imaging (MRI) device, and a computed radiography (CR) device.

The modes of display of the radiologic images appearing on the image display area 10 can be varied by the operation specific to the touch panel 4. For example, one of the radiologic images on the image display area 10 can be enlarged by pinching out or shrunken by pinching in within a partitioned area. Furthermore, the radiologic image can be temporarily hidden from the image display area 10 by flicking or swiping and can be downloaded into the storage 3 in the display terminal 1 by long tapping.

The related examination list display area 20 and the thumbnail display area 30 neighbor to each other and are disposed in the lower portion of the image display area 10 (at the lower end of the display screen).

The related examination list appearing on the related examination list display area 20 is set so that contents of the related examinations are displayed in a chronologically listed state. The related examination list is displayed in a state in which all or parts of the contents of the related examinations are listed. Multiple contents of the related examinations may be displayed or only one content of the related examinations may be displayed.

In more detail, the contents of the related examinations are expressed by characters (character information). In the present embodiment, the content of one related examination includes information regarding indicator, date, and type of modality, which lies side by side. Contents of related examinations including this information are vertically listed in the chronological order in the related examination list display area 20.

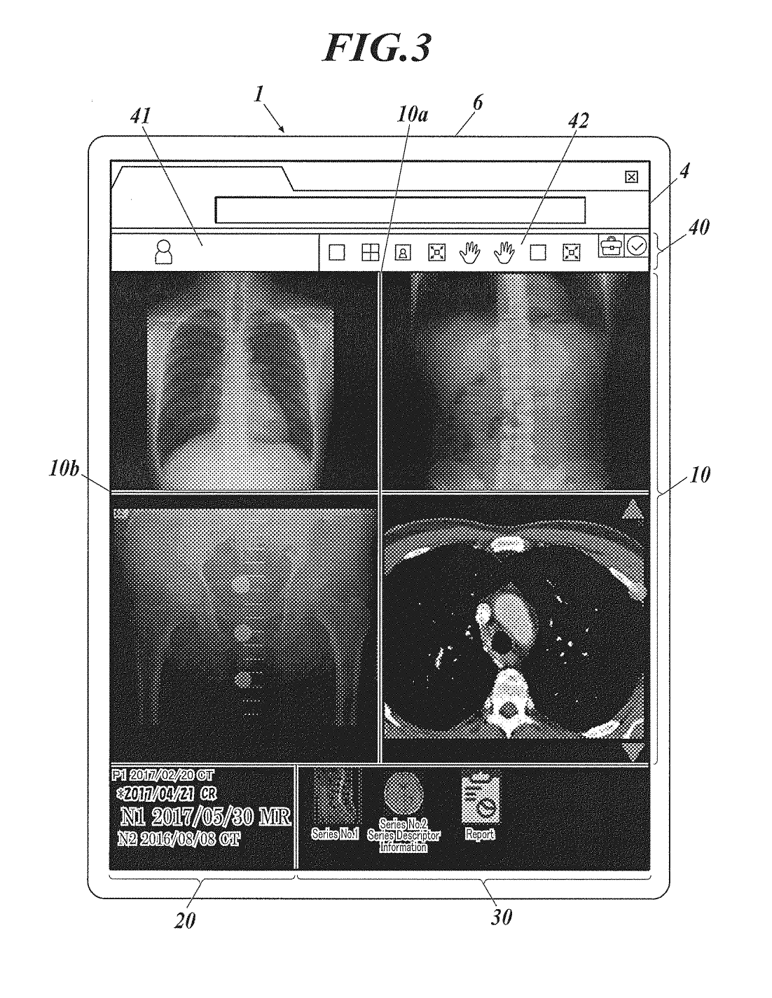

As illustrated in FIG. 2 and FIG. 3, the related examination list display area 20 chronologically exhibits items on the related examination list in series in response to a specific operation with, for example, a finger or a stylus. The contents of related examinations are vertically listed in the order of date and can be thus displayed in the order of date by scrolling down (or flicking down) vertically.

The related examinations displayed as a list in the related examination list display area 20 are list-scrolled like a rotating belt on a drum. In such a mode of scrolling, a related examination appears at the most foreground position and others appear at background positions. In FIG. 2, the content of the present examination appear at the most foreground position. In FIG. 3, the list items are scrolled in the state of FIG. 2, and then a related examination other than the present examination appears at the most foreground position.

FIG. 2 and FIG. 3 illustrate five items in total, i.e., the central current item, two upper items, and two lower items, on the related examination list. In order to enlarge characters of the list items, a total of three list items may be displayed with one list item in the center and the other list items in the lower portion and the higher portion. Alternatively, a single list item may be displayed. Upwardly and downwardly oriented arrows may be additionally displayed to indicate that the related examination list can be scrolled up or down.

The indicator of each related examination is a mark that distinguishes the present examination as a reference from other related examinations.

This indicator shows the temporal anteroposterior relation of the examinations based on when the related examinations on the list were performed. In the present embodiment, the present examination as a reference is indicated with an indicator of “*”. A related examination performed on a date earlier than that of the present examination is indicated with “P” (previous) and a related examination performed on a date later than that of the present examination is indicated with “N” (new). For those near the present examination among the related examinations indicated with “P” and “N”, integers are additionally displayed with “P” and “N”. The past related examination immediately before the present examination is indicated as “P1”, and the new related examination immediately after the present examination is indicated as “N1”. As the time interval from the present examination is long, the suffixed integer is large.

As illustrated in FIG. 2 and others, the types of modality indicated in the contents of the related examinations are alphabetically indicated, such as “CR”, “CT”, and “MR”.

The characters of the contents of the related examinations on the list appear in different display manners based on when the present examination and the related examinations are performed.

In more detail, the characters expressing the contents of the related examinations appear in different display manners, and at least one of the font, color, size, thickness, ornament, angle, character interval, and character width of the character is varied depending on when the examinations are performed (dates). In the present embodiment, the examinations are classified into three types of the present examination, a related examination group dated earlier than the present examination, and a related examination group dated later than the present examination. These three examinations classified according to the dates appear in different display manners of characters.

In the related examination list according to the present embodiment, the content of the present examination is indicated by “*2017/04/21 CR” with a bold pop font and a large size.

The content of the related examination on a date earlier than the present examination (immediately before the present examination) is indicated by “P1 2017/02/20 CT” with a fine gothic font and a small size.

The content of the related examination on a date later than the present examination (immediately after the present examination) is indicated by “N1 2017/05/30 MR” with a fine Ming font and a small size.

In the present embodiment, items on the related examination list are tapped or swiped with, for example, a finger or a stylus to replace the radiologic images in the image display area 10. In other words, tapping of the item of the present examination displays the radiologic image of the present examination. Tapping of an item of the related examination which is not the present examination displays the radiologic image of the related examination.

As illustrated in FIG. 6 and FIG. 7, the display manners of characters representing the pieces 11a to 11h, 12a, and 12b of accompanying information in the image display area 10 and the display manners of characters representing the contents of the related examinations in the related examination list display area 20 are set to be same based on the when the related examinations are performed.

In other words, whether a radiologic image in the image display area 10 and the contents of the related examination list match each other can be confirmed by the display manners of characters.

In FIG. 6, the display manner of the characters of the accompanying information 11a to 11h for all the radiologic images in the image display area 10 is the same as that of the present examination. Thus, it can be confirmed at a glance that all the radiologic images in FIG. 6 are the radiologic images for the present examination.

In FIG. 7, the display manner of characters of the accompanying information 12a and 12b of one of the radiologic images in the image display area 10 is the same as that of the related examination (new related examination immediately after the present examination). Thus, it can be confirmed at a glance that this radiologic image in FIG. 7 is a radiologic image of the related examination (new related examination immediately after the present examination).

The thumbnail display area 30 displays the thumbnails of the radiologic image appear at the most foreground position in the related examination list display area 20. In FIG. 2, the thumbnail display area 30 displays thumbnails of the present examination. In FIG. 3, the thumbnails of a related examination (new related examination immediately after the present examination) are displayed.

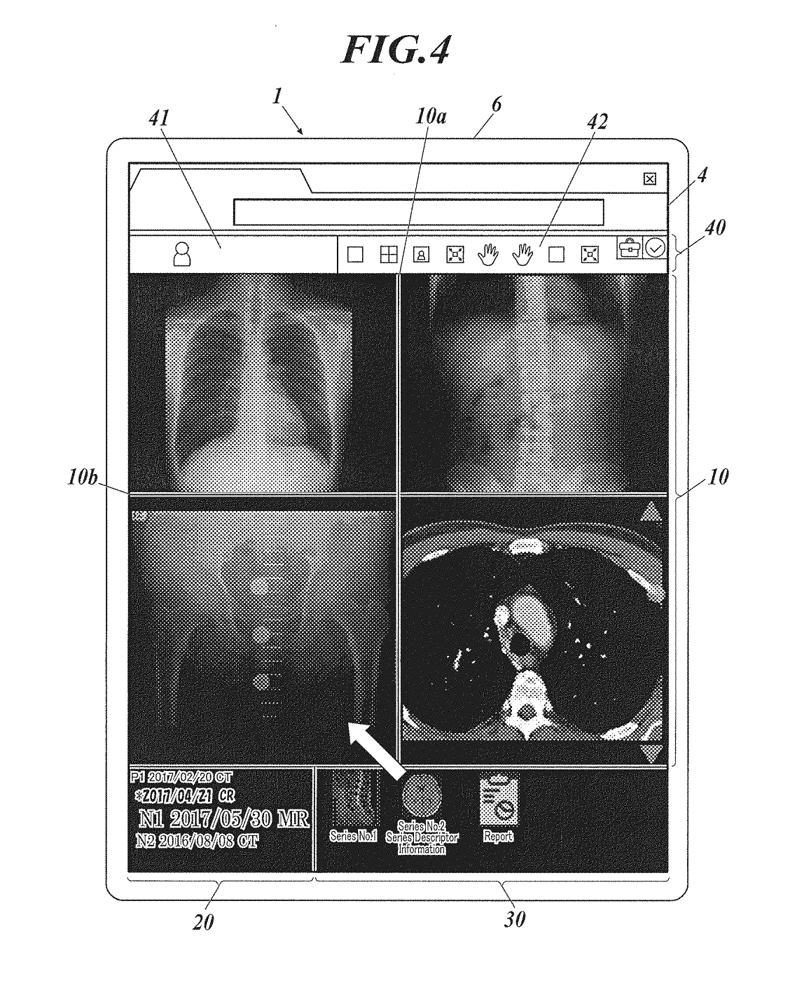

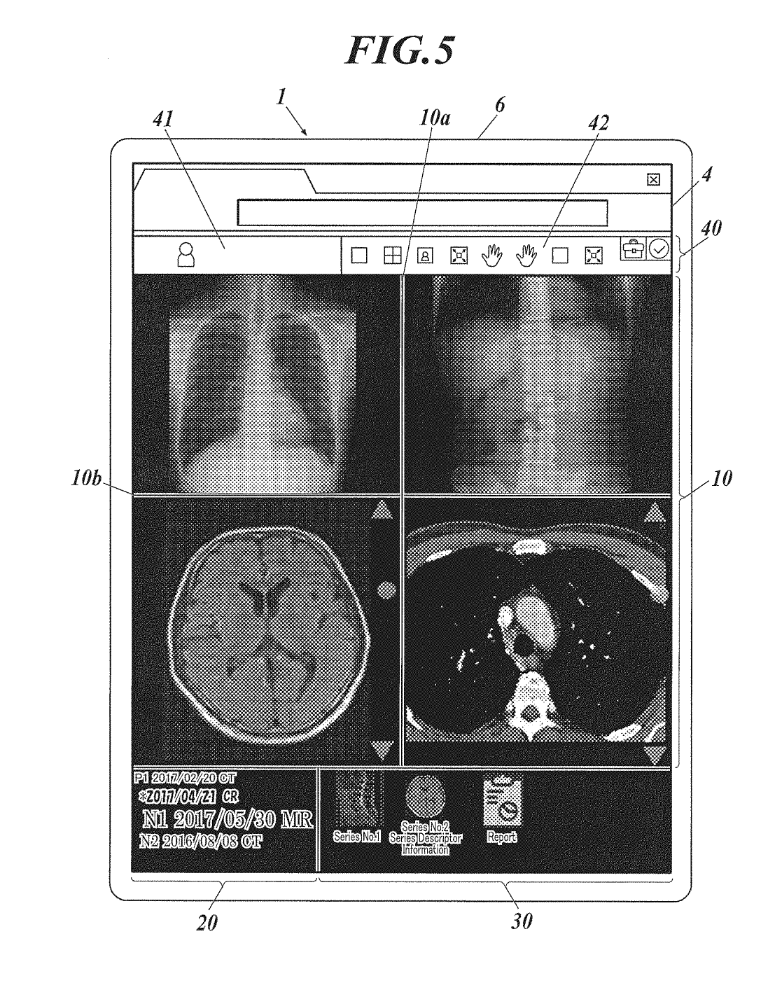

As illustrated in FIG. 4 and FIG. 5, the thumbnails in the thumbnail display area 30 can be dragged into the image display area 10 with, for example, a finger or a stylus. In more detail, any thumbnail in the thumbnail display area 30 of FIG. 4 is touched by a finger or a stylus and is directly drawn into the image display area 10 without releasing the finger or the stylus from the display screen (see the arrow in FIG. 4). Radiologic images can be thereby displayed as illustrated in FIG. 5. FIG. 7 shows an example of the display screen on which display is performed with the same step.

The thumbnail display area 30 can be transversely swiped and thereby transversely scrolled. This can hide a visible thumbnail or display a hidden thumbnail.

The function area 40 includes a patient information display field 41 displaying the information on a patient or subject of a radiologic image in the image display area 10 (for example, patient name and patient ID) and a belt-like toolbar 42 provided with function buttons.

The buttons on the toolbar 42 include a button for switching between the visible state and the hidden state of the pieces 11a to 11h, 12a, and 12b of accompanying information in the image display area 10 and a button for edit and operation of a radiologic image (for example, for enlargement and shrinkage). The buttons can be operated by tapping.

In the case that the button for switching between the visible state and the hidden state of the pieces 11a to 11h, 12a, and 12b of accompanying information in the image display area 10 is tapped for display of the pieces 11a to 11h, 12a, and 12b of accompanying information in the image display area 10, the display manner of characters representing the pieces 11a to 11h, 12a, and 12b of accompanying information are set to be automatically same as that of the contents of related examinations.

The display terminal 1 having such a configuration is not especially limited, but assumed to be usually used in a case where a user, such as a medical doctor, is located away from the in-hospital client terminal and needs to check radiologic images to be interpreted.

The display terminal 1 according to the present embodiment is a tablet computer, but not limited to this. The display terminal 1 may be a more compact terminal, such as a smartphone. In the case of a display terminal smaller than a tablet computer, if the display terminal has a display screen with a layout similar to that of the tablet computer, the displayed radiologic image may be too small and the various operations cannot be performed easily. Thus, in the case that a compact terminal is used as a display terminal 1, it is desirable that the terminal has a display screen with a layout appropriate to the compact display terminal. For example, the image display area 10 may have an unpartitioned layout without the boundaries 10a and 10b.

As described above, according to the present embodiment, there is provided an in-hospital server that is an external device which stores a radiologic image; and a display terminal 1 which displays the radiologic image received from the in-hospital server that is the external device on a display screen of a touch panel 4. The display terminal 1 has, on the display screen, an image display area 10 for display of the radiologic image and a related examination list display area 20 for display of a related examination list to select the radiologic image, and contents of a plurality of related examinations in the related examination list are chronologically displayed in order based on a screen touch operation on the display screen of the touch panel 4. Thus, while displaying the contents of related examinations chronologically in order in the related examination list display area 20 without use of the display layout of an in-hospital monitor, it is possible to properly select a related examination of which radiologic image should be confirmed, and reflect the selection in the image display area 10. Thus, convenient radiologic interpretation can be achieved even on a compact terminal, for example, a smartphone or a tablet computer.

The screen touch operation on the display screen of the touch panel 4 is a screen touch operation to the related examination list display area 20. Thus, in a case of displaying the contents of the related examinations chronologically in order in the related examination list display area 20, the related examination list display area 20 to be operated is directly operated. Thus, an intuitive operation can be achieved.

The listed contents of the related examinations are displayed in different display manners of characters based on when the related examinations are performed. The temporal sequence of the related examinations can be readily grasped from the display manners of characters. Thus, convenient radiologic interpretation can be achieved without use of the layout displayed on the in-hospital monitor.

The image display area 10 displays the radiologic image and pieces 11a to 11h, 12a, and 12b of accompanying information of the radiologic image. It is set that the pieces 11a to 11h, 12a, and 12b of accompanying information appear in the same character display manner as the contents of the related examinations based on the when the related examinations are performed. Thus, it is possible to easily grasp for which related examination the radiologic image is displayed in the image display area 10 among the related examinations. Thus, when the radiologic images of the related examinations are compared and interpreted, the radiologic images can be conveniently interpreted with the reduced confusion of the images.

Each of the listed contents of the related examinations includes an indicator of a temporal anteroposterior relation based on when the related examinations are performed. Thus, for example, the contents of the related examinations in similar display manners of characters can be confirmed by the indicator without depending on the display manners of characters, and the temporal sequence of the related examinations can be readily grasped.

According to the display terminal 1 (image display terminal 1) in the present embodiment, there is provided a display which displays the radiologic image received from the external device on a display screen of a touch panel 4, and there is provided, on the display screen, an image display area 10 for display of the radiologic image and a related examination list display area 20 for display of a related examination list to select the radiologic image, and contents of a plurality of related examinations in the related examination list are chronologically displayed in order based on a screen touch operation to the related examination list display area 20. Thus, while displaying the contents of related examinations chronologically in order in the related examination list display area 20 without use of the display layout of an in-hospital monitor, it is possible to properly select a related examination of which radiologic image should be confirmed, and reflect the selection in the image display area 10. Thus, convenient radiologic interpretation can be achieved even on a compact terminal, for example, a smartphone or a tablet computer.

(Exemplary Configurations)

It should be noted that the embodiment according to the present invention is not limited to the above embodiment, and modifications can be made as needed within the scope of the present invention. Exemplary configurations will now be described. The following exemplary configurations may be combined with each other as far as possible.

(First Configuration)

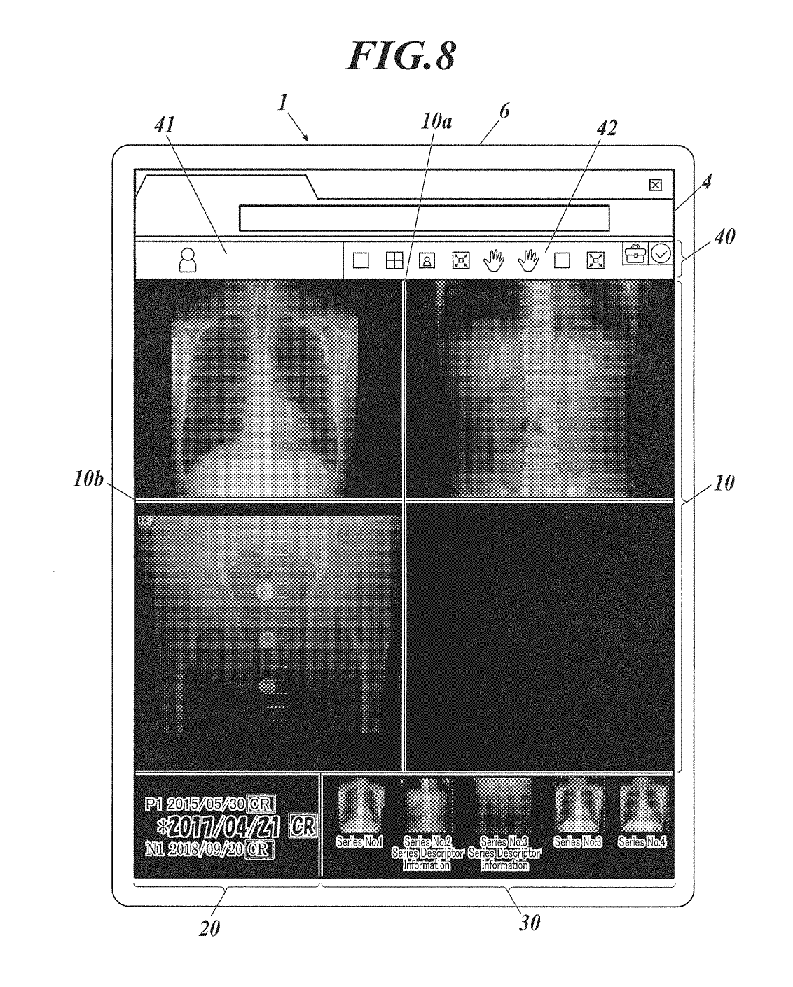

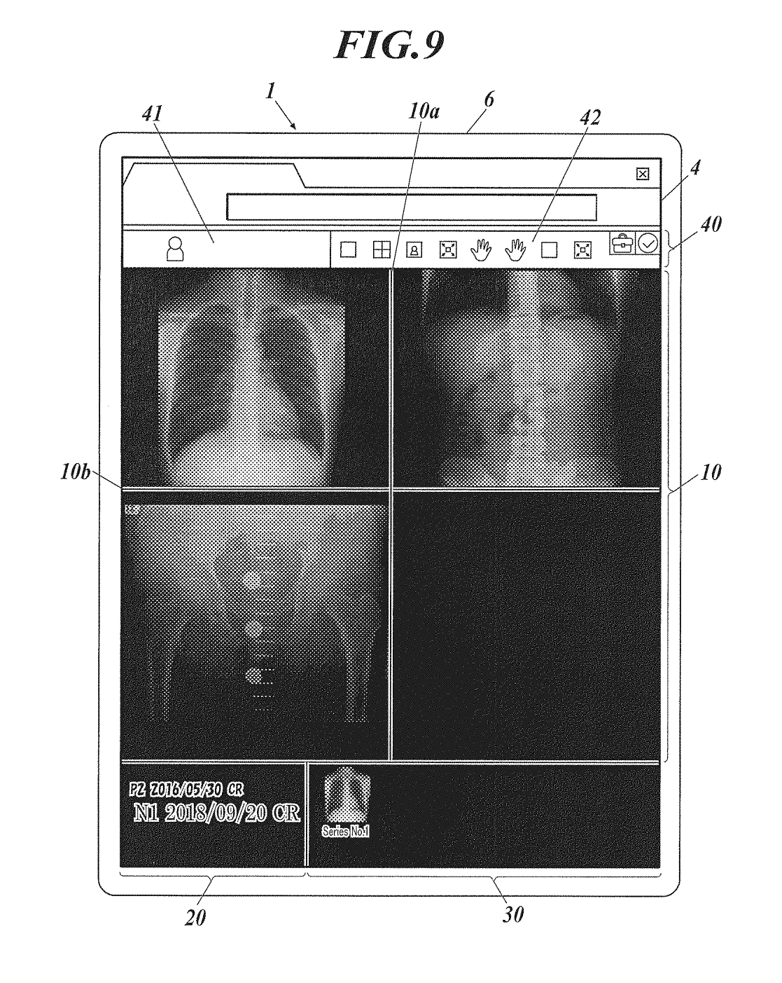

A related examination list according to the first configuration sorts the listed contents of related examinations based on the types of modality or examined sites as illustrated in FIG. 8 and FIG. 9.

The display screen of the display terminal 1 in FIG. 8 is in a state after the portion of alphabetical characters indicating the type of modality is tapped among the contents of the related examinations in the related examination list display area 20.

In FIG. 8, the type of modality “CR” is selected. Alternatively, any other type of modality in the related examination list display area 20 may be selected for sort of the contents of the related examinations.

In such a configuration, the radiologic images of the related examinations can be confirmed for each type of modality, and convenient radiologic interpretation can be achieved.

The display screen of the display terminal 1 in FIG. 9 is in a state after the thumbnail of an arbitrary examined site is tapped among the thumbnails in the thumbnail display area 30.

In FIG. 9, the examined site of “lung field (including the trunk around it) is selected. Alternatively any other examined site in the thumbnail display area 30 may be selected for sort of the contents of the related examinations.

In such a configuration, the radiologic images of the related examinations can be confirmed for each of the examined sites, and convenient radiologic interpretation can be achieved.

(Second Configuration)

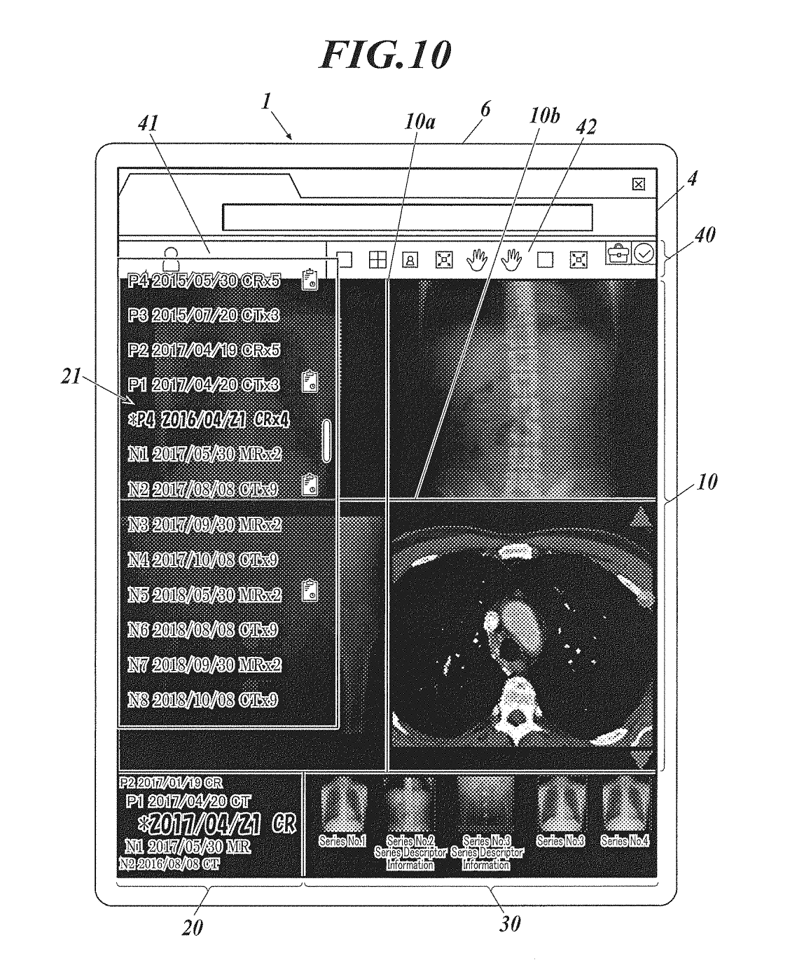

An image display area 10 according to the second configuration displays a sub-related examination list 21 chronologically listing the contents of related examinations as illustrated in FIG. 10.

The sub-related examination list 21 can be displayed in the image display area 10 by tapping of the related examination list display area 20.

The contents of the related examinations on the sub-related examination list 21 are the same as those of the related examinations in the related examination list display area 20 and are used if display of a list of the related examinations is desirable.

Besides the contents, the display manners of the characters expressing the contents of the related examination on the sub-related examination list 21 are the same as those of the related examinations in the related examination list display area 20.

If the number of the related examinations is large and all of them cannot be displayed on the sub-related examination list 21 at a time, the sub-related examination list 21 may be scrollable.

In such a configuration, contents of the related examinations that cannot appear in the related examination list display area 20 at a time can be displayed on the sub-related examination list 21. Thus, the contents of the related examinations can be readily confirmed, and convenient radiologic interpretation can be achieved.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese patent Application No. 2017-247216, filed on Dec. 25, 2017, is incorporated herein by reference in its entirety.

What is claimed is:

1. An image display system comprising:
an external device which stores a radiologic image; and
a display terminal which displays the radiologic image on a display screen of a touch panel, wherein
the display terminal has, on the display screen, an image display area for display of the radiologic image and a related examination list display area for display of a related examination list to select the radiologic image, and
contents of a plurality of related examinations in the related examination list are chronologically displayed in order based on a screen touch operation on the display screen of the touch panel.

2. The image display system according to claim 1, wherein the screen touch operation on the display screen of the touch panel is a screen touch operation to the related examination list display area.

3. The image display system according to claim 1, wherein the listed contents of the related examinations are displayed in different display manners of characters based on when the related examinations are performed.

4. The image display system according to claim 3, wherein
accompanying information of the radiologic image is displayed along with the radiologic image in the image display area, and
a display manner of a character showing the accompanying information and the display manners of the characters expressing the contents of the related examinations are set to be same based on when the related examinations are performed.

5. The image display system according to claim 1, wherein, for the listed contents of the related examinations, an indicator showing a temporal anteroposterior relation is displayed based on when the related examinations are performed.

6. The image display system according to claim 1, wherein the listed contents of the related examinations are sorted based on a type of a modality or an examined site.

7. An image display terminal comprising:
a display that displays a radiologic image, which is received from an external device, on a display screen of a touch panel; and
an image display area for display of the radiologic image and a related examination list display area for display of a related examination list to select the radiologic image, on the display screen, wherein
contents of a plurality of related examinations in the related examination list are chronologically displayed in order based on a screen touch operation to the related examination list display area.
